(19) United States
(12) Patent Application Publication
(10) Pub. No.: US 2026/0077166 A1
(43) Pub. Date: Mar. 19, 2026

(54) METHOD FOR MANUFACTURING MEDICAL DEVICE AND MEDICAL DEVICE

(71) Applicant: ASAHI INTECC CO., LTD, Seto-shi (JP)

(72) Inventor: Takeru SATO, Seto-shi (JP)

(73) Assignee: ASAHI INTECC CO., LTD, Seto-shi (JP)

(21) Appl. No.: 19/332,983

(22) Filed: Sep. 18, 2025

(30) Foreign Application Priority Data
Sep. 18, 2024 (JP) 2024-160687

(51) Int. Cl. A61M 25/09

(52) U.S. Cl. CPC A61M 25/09041; A61M 2025/09108; A61M 2025/09175

(57) ABSTRACT

A method for manufacturing a medical device includes: manufacturing an entry member entering a lesion by forming a through-hole in a flat portion of a workpiece having the flat portion, the entry member having a loop portion surrounding the through-hole; and joining the entry member and an elongated body to each other in such a way that the through-hole is oriented in a predetermined direction intersecting an axial direction of the elongated body.

### CROSS REFERENCE TO RELATED APPLICATIONS

This is a Paris Convention application based on JP 2024-160687 filed Sep. 18, 2024. The disclosure of the prior application is hereby incorporated by reference herein in its entirety.

### TECHNICAL FIELD

The technology disclosed herein relates to a method for manufacturing a medical device and a medical device.

### BACKGROUND ART

Known medical devices include a loop portion at a distal end portion. The loop portion is formed by folding back an elongated member (for example, see Patent Document 1).

### CITATION LIST

Patent Document 1: Japanese Unexamined Patent Publication No. 2006-507899

### SUMMARY

### Technical Problem

There is room for improvement in the strength of the loop portion in the medical device.

The present specification discloses a technology capable of solving the above-described problem.

### Solution to Problem

The technology disclosed in this specification can be achieved, for example, as the following aspect.

A method for manufacturing a medical device disclosed in this specification includes: manufacturing an entry member entering a lesion by forming a through-hole in a flat portion of a workpiece having the flat portion, the entry member having a loop portion surrounding the through-hole; and joining the entry member and an elongated body in such a way that the through-hole is oriented in a predetermined direction intersecting an axial direction of the elongated body.

### BRIEF DESCRIPTION OF DRAWINGS

FIG. 1 is an explanatory view schematically illustrating a configuration of a guide wire according to a first embodiment.

FIG. 2 is an explanatory view schematically illustrating a configuration of the guide wire according to the first embodiment.

FIG. 3 is an explanatory view schematically illustrating a configuration of the guide wire according to the first embodiment.

FIG. 4 is an explanatory view schematically illustrating a configuration of the guide wire according to the first embodiment.

FIG. 5 is an explanatory view schematically illustrating a configuration of the guide wire according to the first embodiment.

FIG. 6 is an explanatory view schematically illustrating a configuration of the guide wire according to the first embodiment.

FIG. 7 is a flowchart illustrating an example of a treatment method using the guide wire.

FIG. 8 is an explanatory view illustrating an example of the treatment method using the guide wire.

FIG. 9 is an explanatory view illustrating an example of the treatment method using the guide wire.

FIG. 10 is an explanatory view illustrating an example of the treatment method using the guide wire.

FIG. 11 is a flowchart illustrating an example of a method for manufacturing the guide wire.

FIG. 12 is an explanatory view illustrating an example of the method for manufacturing the guide wire.

FIG. 13 is an explanatory view schematically illustrating a configuration of a guide wire according to a second embodiment.

FIG. 14 is an explanatory view schematically illustrating a configuration of a guide wire according to a third embodiment.

FIG. 15 is an explanatory view schematically illustrating a configuration of a guide wire according to a fourth embodiment.

FIG. 16 is an explanatory view schematically illustrating a configuration of a guide wire according to a fifth embodiment.

FIG. 17 is an explanatory view schematically illustrating a configuration of a guide wire according to a sixth embodiment.

FIG. 18 is an explanatory view schematically illustrating a configuration of a guide wire according to a seventh embodiment.

FIG. 19 is an explanatory view schematically illustrating a configuration of a guide wire according to an eighth embodiment.

FIG. 20 is an explanatory view schematically illustrating a configuration of a guide wire according to a ninth embodiment.

FIG. 21 is an explanatory view schematically illustrating a configuration of a guide wire according to a tenth embodiment.

FIG. 22 is an explanatory view schematically illustrating a configuration of a guide wire according to a modification.

### DETAILED DESCRIPTION

### A. First Embodiment

### Basic Configuration of Guide Wire 100

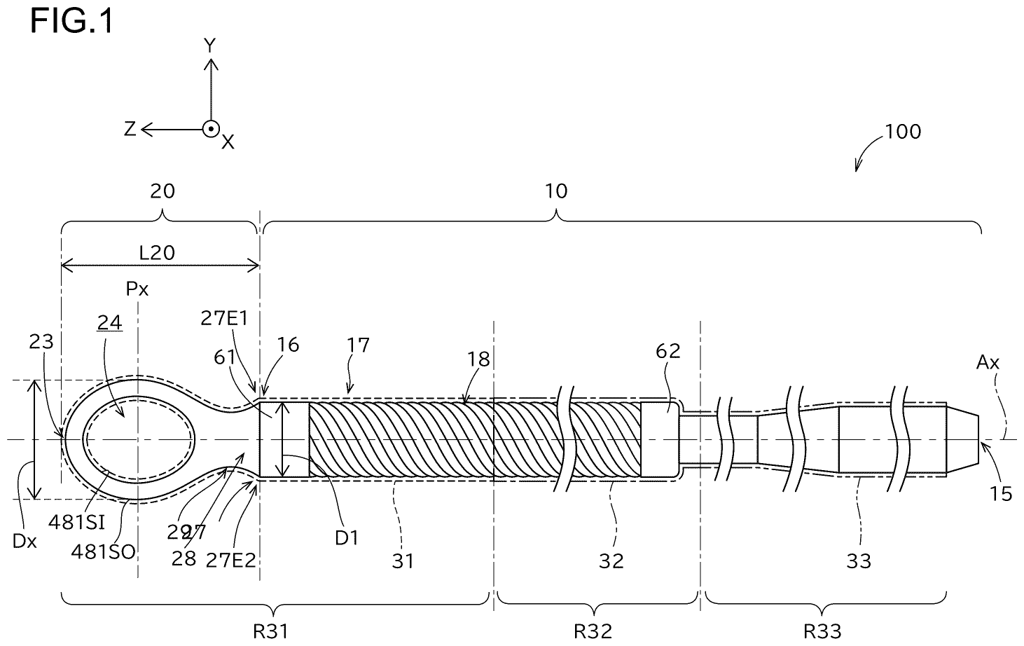

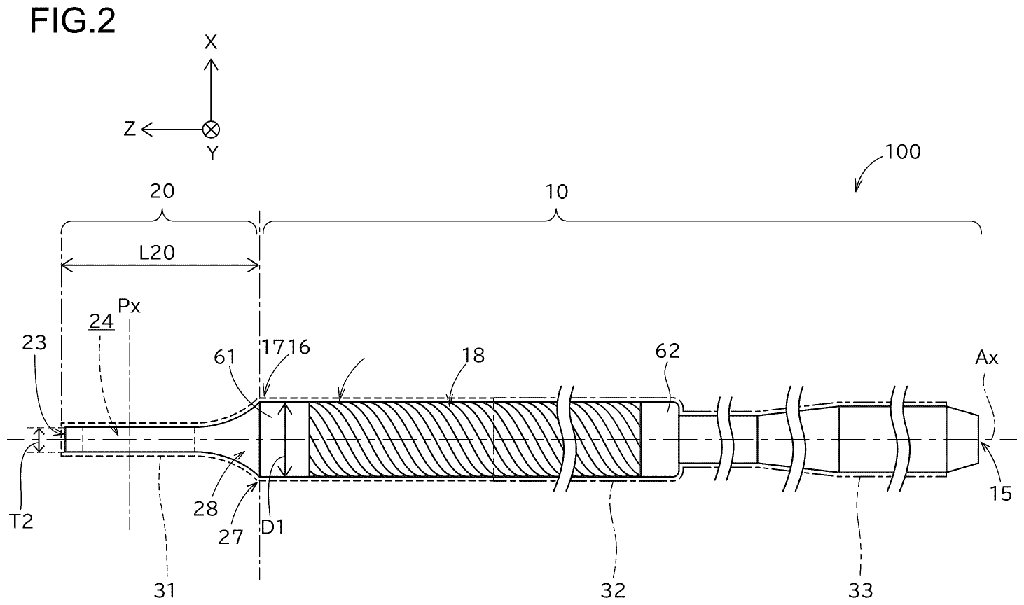

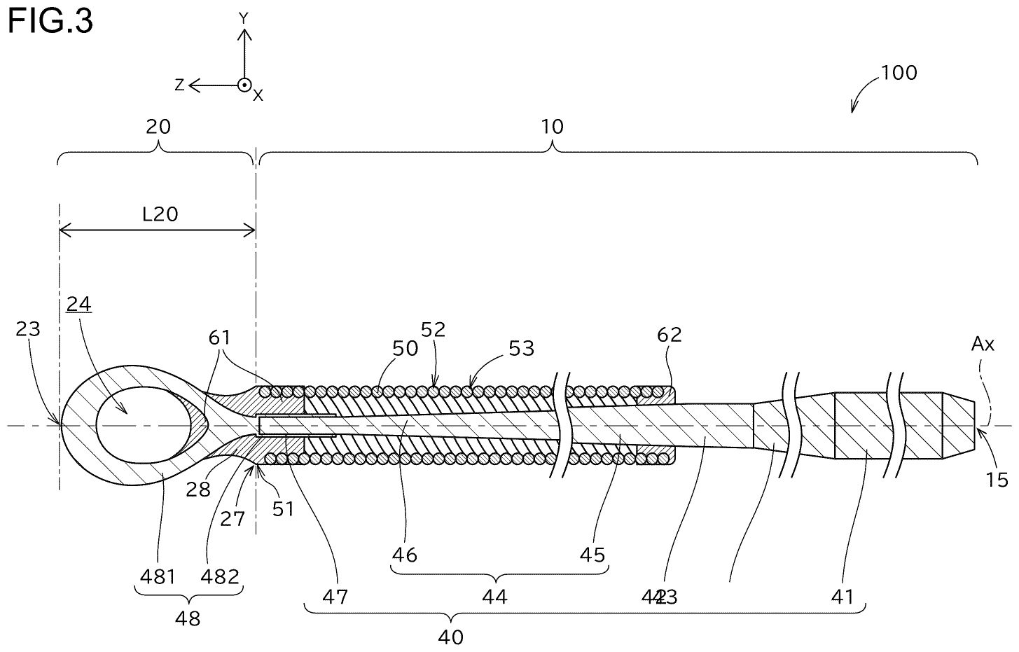

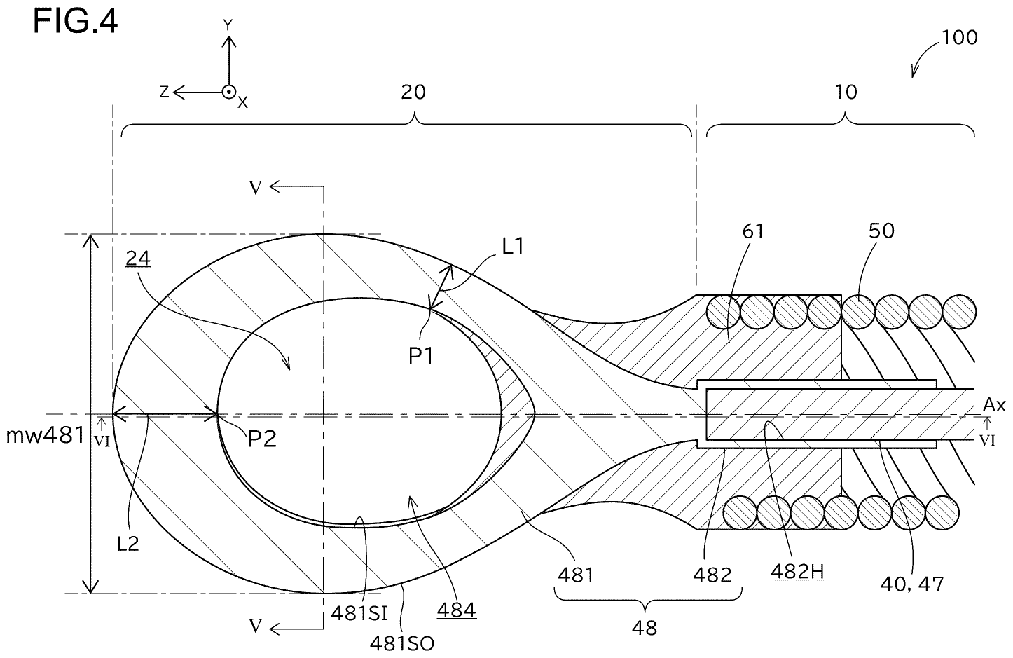

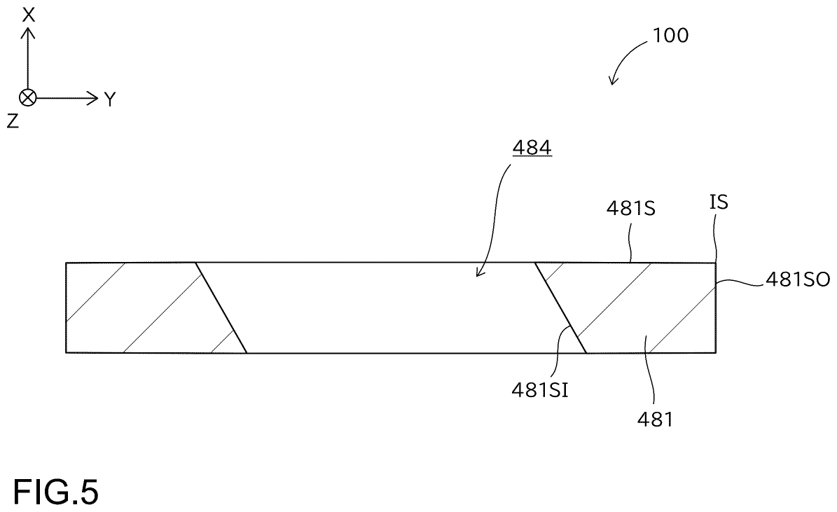

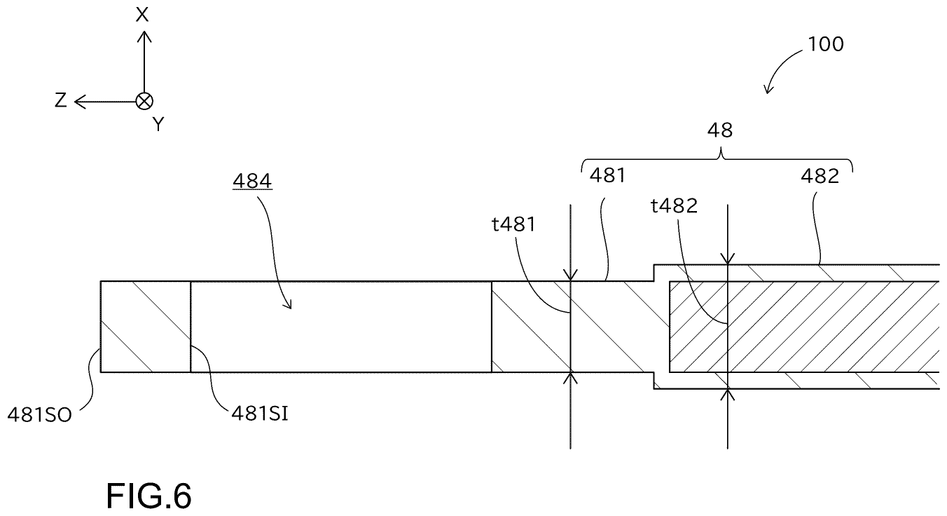

FIGS. 1 to 6 are explanatory views schematically illustrating a configuration of a guide wire 100 according to a first embodiment. In each drawing, XYZ axes orthogonal to each other for specifying a direction are illustrated. FIG. 1 illustrates an external appearance of the guide wire 100 as viewed in an X-axis direction. FIG. 2 illustrates an external appearance of the guide wire 100 as viewed in a Y-axis direction. FIG. 3 illustrates a YZ longitudinal section of the guide wire 100. FIG. 4 illustrates a YZ longitudinal section of a distal end portion of the guide wire 100. FIG. 5 illustrates an XY transverse section of the guide wire 100 at a position V-V in FIG. 4. FIG. 6 illustrates an XZ longitudinal section of the guide wire 100 at a position VI-VI in FIG. 4. In the guide wire 100, a Z-axis positive direction side is a distal end side (far side) to be inserted into a body. In the guide wire 100, a Z-axis negative direction side is a proximal end side (near side) to be operated by a professional. In each drawing, a part of the guide wire 100 may be omitted. FIGS. 1 to 3 illustrate a state where the guide wire 100 is in a linear shape parallel to the Z-axis. The guide wire 100 is flexible enough to be curved. These points are also the same in the subsequent drawings. The X-axis direction is an example of a predetermined direction.

In the present specification, regarding the guide wire 100 and each component thereof, an end on the distal end side is referred to as a “distal end”, the distal end and the vicinity thereof are referred to as a “distal end portion”, an end on the proximal end side is referred to as a “proximal end”, and the proximal end and the vicinity thereof are referred to as a “proximal end portion”. A transverse section of the guide wire 100 and each component thereof refers to a cross section orthogonal to a longitudinal direction. A longitudinal section of the guide wire 100 and each component thereof refers to a cross section parallel to a center axis in the longitudinal direction. Regarding the guide wire 100 and each component thereof, a direction orthogonal to the longitudinal direction is referred to as a “radial direction”. An outer diameter of the guide wire 100 and each component thereof refers to a length along the radial direction. In the present specification, regarding the guide wire 100 and each component thereof, a length along the Y-axis direction may be particularly referred to as a “width”, and a length along the X-axis direction may be particularly referred to as a “thickness”.

The guide wire 100 is an elongated medical device to be inserted into a living body lumen such as a blood vessel. An entire length of the guide wire 100 is, for example, not less than 1000 mm and not more than 3000 mm. The guide wire 100 is an example of the medical device.

The guide wire 100 includes a main body part 10 and a leading portion 20.

The main body part 10 is an elongated portion extending along a center axis Ax. In the present embodiment, the center axis Ax of the main body part 10 coincides with the center axis of the guide wire 100. A proximal end 15 of the main body part 10 coincides with the proximal end of the guide wire 100. A spiral groove 18 is formed on an outer peripheral surface 17 of the distal end portion of the main body part 10.

The leading portion 20 is connected to a distal end 16 of the main body part 10. A proximal end 27 of the leading portion 20 is connected to the distal end 16 of the main body part 10. The leading portion 20 can be expressed as a guiding portion, a drilling portion, a fracturing portion, a peeling portion, an advancing portion, a peeler, a shaver, or the like. A distal end 23 of the leading portion 20 coincides with the distal end of the guide wire 100. The leading portion 20 has a loop shape surrounding a through-hole 24 extending in the X-axis direction. A surface of the leading portion 20 may have an edge or may not have an edge. The edge is a boundary (ridge line) between two surfaces. The leading portion 20 enters a lesion while rotating around the center axis Ax. The entry of the leading portion 20 into the lesion can be expressed as crossing/passing through the lesion, drilling the lesion, fracturing the lesion, peeling the lesion, pushing through the lesion, diving into the lesion, advancing into the lesion, and the like. A length L20 of the leading portion 20 along the center axis Ax is, for example, not less than 0.2 mm and not more than 2.0 mm. The length L20 of the leading portion 20 may be not less than 0.3 mm and not more than 1.5 mm, or may be not less than 0.4 mm and not more than 1.0 mm. When viewed in the X-axis direction, an end portion of the proximal end 27 of the leading portion 20 on a Y-axis positive direction side is referred to as a first end portion 27E1, and an end portion of the proximal end 27 of the leading portion 20 on a Y-axis negative direction side is referred to as a second end portion 27E2.

A maximum outer diameter Dx of the leading portion 20 is larger than a thickness T2 of the leading portion 20 at a maximum outer diameter position Px. That is, the leading portion 20 has a flat shape as a whole. The thickness T2 of the leading portion 20 at the maximum outer diameter position Px is smaller than a maximum outer diameter D1 of the distal end 16 of the main body part 10. The thickness T2 of the leading portion 20 at the maximum outer diameter position Px is, for example, not less than 0.02 mm and not more than 0.3 mm. The thickness T2 of the leading portion 20 at the maximum outer diameter position Px may be not less than 0.04 mm and not more than 0.2 mm, or may be not less than 0.06 mm and not more than 0.1 mm. The maximum outer diameter Dx of the leading portion 20 is, for example, not less than 1.2 times the thickness T2 of the leading portion 20 at the maximum outer diameter position Px. The maximum outer diameter Dx of the leading portion 20 may be not less than 1.5 times or not less than 1.8 times the thickness T2 of the leading portion 20 at the maximum outer diameter position Px.

The maximum outer diameter Dx of the leading portion 20 is measured as follows. A measurer observes the guide wire 100 from a side thereof. The side is the Y-axis direction in the present embodiment. The measurer searches for an angle at which a portion on the near side and a portion on the far side of the leading portion 20 overlap each other and the portion on the far side cannot be seen. For example, when both of the following two conditions are satisfied, the measurer searches for an angle at which a portion between the second end portion 27E2 and the distal end 23 cannot be seen. The first condition is that a portion between the first end portion 27E1 and the distal end 23 is located on the near side. The second condition is that the portion between the second end portion 27E2 and the distal end 23 is located on the far side. The invisibility of the portion between the second end portion 27E2 and the distal end 23 is caused by an overlap between the portion between the first end portion 27E1 and the distal end 23 and the portion between the second end portion 27E2 and the distal end 23. Next, the measurer photographs the guide wire 100 by using a microscope along a viewpoint rotated by 90 degrees around the center axis Ax from the viewpoint at this time. The measurer sets a photographing magnification of the microscope to 200 times or more. The measurer measures the outer diameter of the leading portion 20 at three measurement positions where the leading portion 20 is considered to have the maximum outer diameter Dx on the captured image. To be specific, at each measurement position, the measurer draws a pair of straight lines in parallel to each other, which pass through a pair of end portions of the leading portion 20 in an outer diameter direction and are orthogonal to the outer diameter direction, and measures an interval between the pair of straight lines. The measurer adopts a maximum value of measurement results at the three measurement positions as the maximum outer diameter Dx of the leading portion 20.

### Treatment Method Using Guide Wire 100

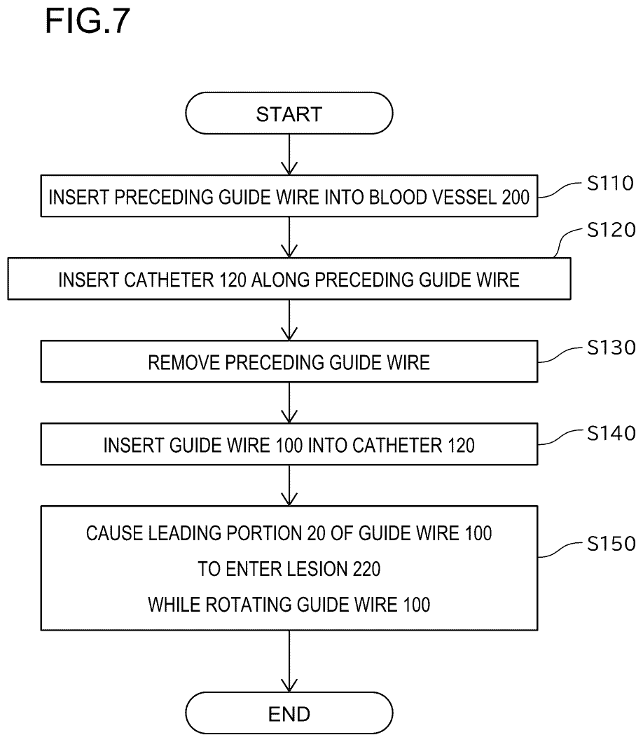

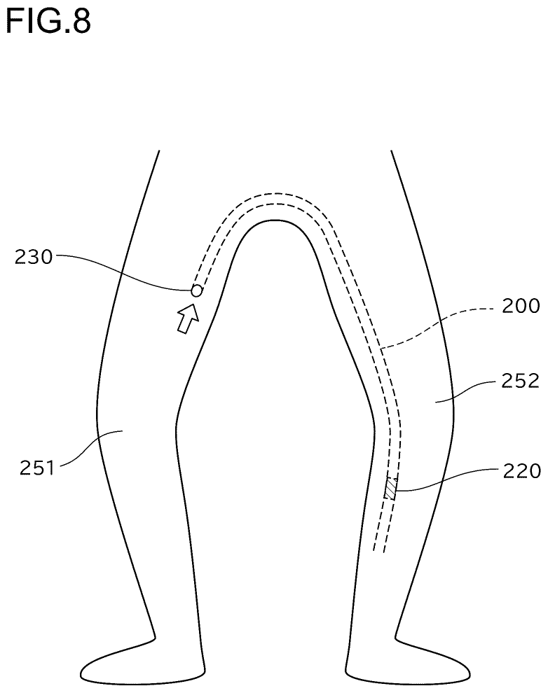

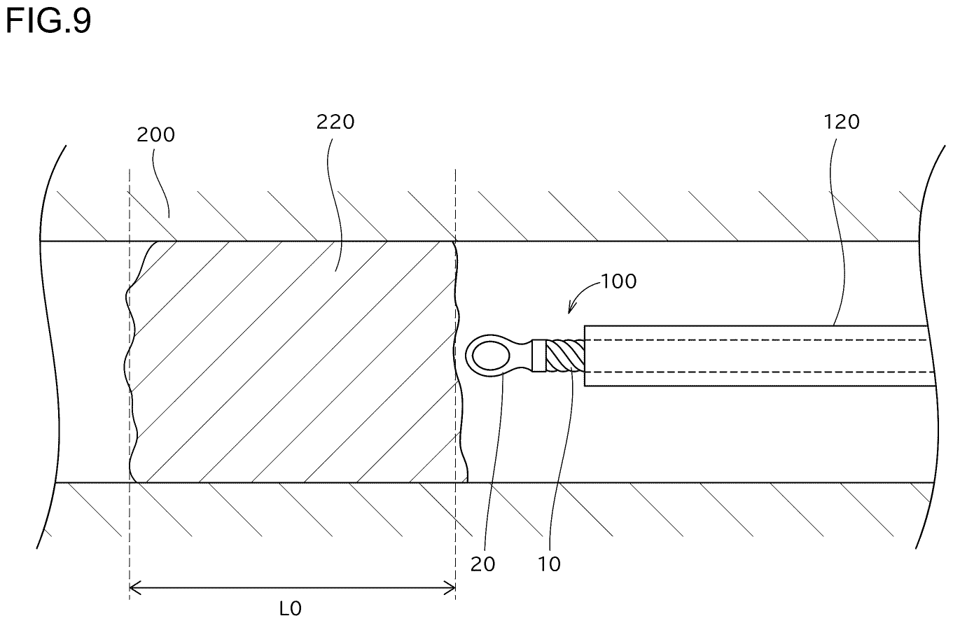

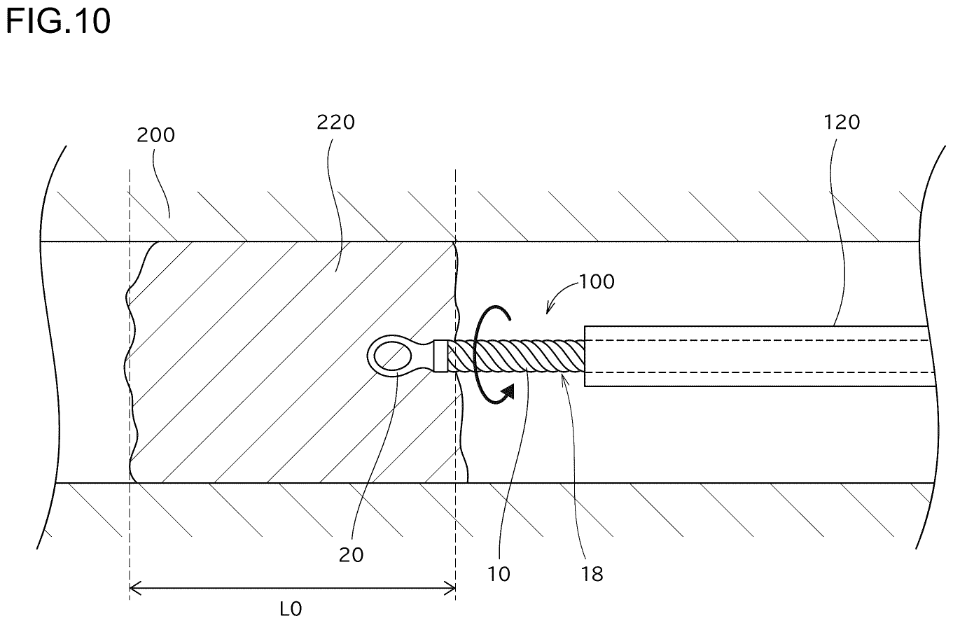

FIG. 7 is a flowchart illustrating an example of a treatment method using the guide wire 100. FIGS. 8 to 10 are explanatory views illustrating an example of the treatment method using the guide wire 100. As illustrated in FIGS. 9 and 10, in the treatment method using the guide wire 100, a professional causes the leading portion 20 of the guide wire 100 to enter a lesion 220 in a blood vessel 200. The lesion 220 is, for example, a highly calcified lesion. The lesion 220 is, for example, a chronic completely occlusion lesion. A length L0 of the lesion 220 along the extending direction of the blood vessel 200 is, for example, not less than 100 mm and not more than 500 mm. The length of the lesion 220 may be not less than 150 mm and not more than 450 mm, or not less than 200 mm and not more than 400 mm.

As illustrated in FIG. 8, the lesion 220 to be treated is located, for example, in the blood vessel 200 of the lower limb of a human. FIG. 8 illustrates a lesion 220 that has occurred in a below-knee region. In this treatment method, for example, a technology called crossover is used in which the blood vessel 200 is accessed from a groin of an opposite leg 251, which is the leg opposite to a target leg 252 in which the lesion 220 is located, and the lesion 220 in the target leg 252 is approached. A method for approaching the lesion 220 that has occurred in the below-knee region by inserting the guide wire 100 into the blood vessel 200 may be a method other than the crossover. The approach method may be an antegrade approach in which the guide wire 100 is inserted through the blood vessel 200 in a groin of the target leg 252 in which the lesion 220 is located, and the guide wire 100 is advanced along the flow of the blood stream. The approach method may be an antegrade approach in which the guide wire 100 is inserted through the blood vessel 200 in the arm and the guide wire 100 is advanced along the flow of the blood stream. The approach method may be a retrograde approach in which the guide wire 100 is inserted through either the blood vessel 200 in the ankle or the blood vessel 200 in the dorsum of the foot, of the target leg 252 in which the lesion 220 is located, and the guide wire 100 is advanced against the flow of the blood stream. The position at which the guide wire 100 is inserted into the blood vessel 200 is not limited to the above-described position. When the guide wire 100 is inserted through the blood vessel 200 in the arm, the professional can select the blood vessel 200 in the wrist as a position at which the guide wire 100 is inserted into the blood vessel 200. When the guide wire 100 is inserted through the blood vessel 200 in the leg, the professional can select a superficial femoral artery or a popliteal artery as the position at which the guide wire 100 is inserted into the blood vessel 200. The guide wire 100 is not limited to the treatment of the lesion 220 that has occurred in the below-knee region, and can be used in the treatment of the lesion 220 that has occurred in an other region such as an iliac artery.

First, the professional inserts a preceding guide wire into the blood vessel 200 (S110). Unlike the guide wire 100 of the present embodiment, the preceding guide wire is a known guide wire that does not have the leading portion 20. The preceding guide wire may be referred to as a workhorse guide wire, a first choice guide wire, or the like. The professional inserts the preceding guide wire into the blood vessel 200 via a sheath (not illustrated) disposed at a puncture position 230 (see FIG. 8) of the opposite leg 251. The professional advances the preceding guide wire to a location immediately proximal to the lesion 220 in the blood vessel 200 of the target leg 252.

Next, the professional inserts a catheter 120 into the blood vessel 200 along the preceding guide wire (S120). The professional advances the catheter 120 (see FIG. 9) to a location immediately proximal to the lesion 220 in the blood vessel 200.

Next, the professional removes the preceding guide wire from the blood vessel 200 (S130). Thereafter, the professional inserts the guide wire 100 into the catheter 120 inserted into the blood vessel 200, with the leading portion 20 at the front (S140, FIG. 9). The professional advances the guide wire 100 to a location immediately proximal to the lesion 220 in the blood vessel 200. When advancing the guide wire 100, the professional may or may not rotate the guide wire 100 around the center axis Ax.

Next, while rotating the guide wire 100, the professional advances the guide wire 100 toward the distal end side, thereby causing the leading portion 20 of the guide wire 100 to enter the lesion 220 (S150, FIG. 10). When the professional grips the proximal end portion of the guide wire 100 and rotates the guide wire 100 around the center axis Ax, the leading portion 20 located at the distal end portion of the guide wire 100 also rotates around the center axis Ax. The leading portion 20, which rotates inside the lesion 220, drills the lesion 220 in a way that tears apart the lesion. In the present embodiment, the professional advances the leading portion 20 until the leading portion 20 passes through the lesion 220 in the step of advancing the guide wire 100 (S150). In a case where the leading portion 20 has an edge, when the leading portion 20 rotates around the center axis Ax in a state where the edge is in contact with the lesion 220, the edge scrapes the lesion 220. The leading portion 20 advances into a space formed by the edge scraping the lesion 220, thereby forming a through-hole in the lesion 220. As described above, the spiral groove 18 is formed on the outer peripheral surface 17 of the main body part 10. Due to the presence of the groove 18, the main body part 10 has a function of discharging small pieces of the lesion 220 generated by contact between the leading portion 20, which is rotating, and the lesion 220 from the distal end side toward the proximal end side of the main body part 10 (referred to as “lesion discharge function”). The step of advancing the guide wire 100 (S150) is performed in a state where no other medical devices have passed through the lesion 220.

After the leading portion 20 of the guide wire 100 has passed through the lesion 220, the professional advances a catheter (not illustrated) to the position of the lesion 220 along the guide wire 100. Thereafter, the professional removes the guide wire 100. When removing the guide wire 100, the professional may or may not rotate the guide wire 100 around the center axis Ax.

Thereafter, the professional inserts a guide wire for an adjunctive device (not illustrated) into the blood vessel 200 and advances the guide wire for an adjunctive device until the distal end of the guide wire for an adjunctive device passes through the lesion 220. The professional advances the adjunctive device to the position of the lesion 220 by following along the guide wire for the adjunctive device. The adjunctive device may be, for example, an atherectomy device, a balloon catheter, a stent, and the like.

### Detailed Configuration of Guide Wire 100

As illustrated in FIG. 3, the guide wire 100 includes a first wire 48, a second wire 40, and a coil 50.

The coil 50 is a cylindrical member in which one or more wires are spirally wound. The main body part 10 includes the coil 50. An outer diameter of the coil 50 is, for example, not less than 0.1 mm and not more than 0.6 mm. The outer diameter of the coil 50 may be not less than 0.2 mm and not more than 0.5 mm, or may be not less than 0.3 mm and not more than 0.4 mm. The outer diameter of the coil 50 may be not less than 1.00 mm and not more than 2.00 mm, may be not less than 1.10 mm and not more than 1.65 mm, or may be not less than 1.20 mm and not more than 1.35 mm. In the present embodiment, the outer diameter of the coil 50 is constant over the entire length of the coil 50. The coil 50 may have a tapered shape in which the outer diameter of the coil 50 gradually decreases from the proximal end toward the distal end, or may have a tapered shape in which the outer diameter of the coil 50 gradually decreases from the distal end toward the proximal end. A spiral groove 53 is formed on an outer peripheral surface 52 of the coil 50. In the present embodiment, the coil 50 is Z-wound, and the running direction of the groove 53 is also a direction of Z-winding. Due to the presence of the spiral groove 53, the spiral groove 18 is formed on the outer peripheral surface 17 of the main body part 10. The distal end of the coil 50 coincides with the distal end 16 of the main body part 10. The coil 50 is an example of an elongated body.

The wire forming the coil 50 may be a single strand or a twisted wire formed by twisting a plurality of strands. In the present embodiment, the coil 50 is a multi-thread coil formed by winding a plurality of wires. In the present embodiment, each wire forming the coil 50 is a twisted wire.

As a material forming the coil 50, for example, a metal is used. More specifically, for example, radiolucent materials such as stainless steels (SUS302, SUS304, SUS316, etc.), Ni-Ti alloys, piano wire, and radiopaque materials such as platinum, gold, tungsten, and any of alloys thereof may be used. The coil 50 may be wholly formed of the same material, or individual portions may each be formed of different materials.

The first wire 48 is a linear member. The leading portion 20 includes the first wire 48. In the present embodiment, the main body part 10 includes a part of the first wire 48 on the proximal end side. The first wire 48 is a member that enters the lesion 220 in treatment using the guide wire 100. The first wire 48 is an example of an entry member.

The first wire 48 includes a loop portion 481 and a base portion 482. The loop portion 481 is an annular portion located on the distal end side of the first wire 48. The loop portion 481 has a flat shape. A maximum width mw481 of the loop portion 481 as viewed in the X-axis direction is longer than a thickness t481 of the loop portion 481 as viewed in the Y-axis direction. A through-hole 484 is formed in the first wire 48. The through-hole 484 passes through the first wire 48 in the X-axis direction intersecting the axial direction of the main body part 10. The loop portion 481 surrounds the through-hole 484. The through-hole 484 forms at least a part of the through-hole 24 of the leading portion 20. The base portion 482 is a portion extending from the proximal end of the loop portion 481 to the proximal end side. As illustrated in FIG. 6, the thickness t481 of the loop portion 481 in the X-axis direction is smaller than a thickness t482 of the base portion 482 in the X-axis direction. A ratio between the thickness t481 and the thickness t482, t481/t482, is, for example, not less than 0.2 and not more than 0.95. t481/t482 may be not less than 0.4 and not more than 0.9, or may be not less than 0.5 and not more than 0.8. The thickness t481 of the loop portion 481 in the X-axis direction may be larger than the thickness t482 of the base portion 482 in the X-axis direction, or may be equal to the thickness t482 of the base portion 482 in the X-axis direction. A hole 482H extending in the axial direction of the main body part 10 is formed in the base portion 482.

As a material forming the first wire 48, for example, a metal is used. In the present embodiment, as a material forming the first wire 48, for example, a radiopaque material such as platinum, gold, tungsten, tantalum, rhenium, iridium, or an alloy thereof is used. That is, in the present embodiment, the first wire 48 is radiopaque. As the material forming the first wire 48, for example, radiolucent materials such as stainless steels (SUS302, SUS304, SUS316, etc.), Ni-Ti alloys, and piano wires may be used. The first wire 48 may be wholly formed of the same material, or individual portions may each be formed of different materials.

The first wire 48 is inserted into a hollow of the coil 50. The coil 50 covers a part of the proximal end side of the first wire 48.

The second wire 40 is a linear member. The main body part 10 includes the second wire 40. The second wire 40 is located on the proximal end side with respect to the first wire 48. A distal end portion of the second wire 40 is connected to a proximal end portion of the first wire 48. The proximal end of the second wire 40 is located at the proximal end portion of the main body part 10. The proximal end side of the second wire 40 is a portion to be gripped by the professional.

The second wire 40 includes a large diameter portion 41, a first tapered portion 42, an intermediate diameter portion 43, a second tapered portion 44, and a small diameter portion 47. In the second wire 40, the large diameter portion 41, the first tapered portion 42, the intermediate diameter portion 43, the second tapered portion 44, and the small diameter portion 47 are arranged in this order from the proximal end side toward the distal end side of the guide wire 100.

The large diameter portion 41 is a rod-shaped portion having a substantially constant outer diameter. The outer diameter (maximum width) of the large diameter portion 41 is, for example, about not less than 0.2 mm and not more than 3.0 mm. The first tapered portion 42 is a portion whose diameter gradually decreases from a boundary with the large diameter portion 41 toward a boundary with the intermediate diameter portion 43. The intermediate diameter portion 43 is a rod-shaped portion having a substantially constant outer diameter smaller than the outer diameter of the large diameter portion 41. The second tapered portion 44 is a portion whose diameter gradually decreases from the boundary with the intermediate diameter portion 43 toward a boundary with the small diameter portion 47. In the present embodiment, the second tapered portion 44 includes a proximal-end-side second tapered portion 45 and a distal-end-side second tapered portion 46 located on the distal end side with respect to the proximal-end-side second tapered portion 45. The small diameter portion 47 is a rod-shaped portion having a substantially constant outer diameter smaller than the outer diameter of the intermediate diameter portion 43.

In the present embodiment, rates of change (hereinafter, referred to as “gradients”) of the outer diameters along the longitudinal direction in the proximal-end-side second tapered portion 45 and the distal-end-side second tapered portion 46 are different from each other. For example, the gradient of the proximal-end-side second tapered portion 45 is steeper than the gradient of the distal-end-side second tapered portion 46. The gradient of the proximal-end-side second tapered portion 45 may be gentler than the gradient of the distal-end-side second tapered portion 46, or may be the same as the gradient of the distal-end-side second tapered portion 46. In the present embodiment, gradients of the first tapered portion 42 and the second tapered portion 44 are different from each other. For example, the gradient of the first tapered portion 42 is steeper than the gradient of the second tapered portion 44. The gradient of the first tapered portion 42 may be gentler than the gradient of the second tapered portion 44, or may be the same as the gradient of the second tapered portion 44.

As a material forming the second wire 40, for example, a metal is used. More specifically, for example, stainless steels (SUS302, SUS304, SUS316, etc.), Ni-Ti alloys, piano wire, and the like are used. The second wire 40 may be wholly formed of the same material, or individual portions may each be formed of different materials. The second wire 40 may be formed of the same material as the first wire 48, or may be formed of a material different from the first wire 48.

The second wire 40 is inserted into the hollow of the coil 50. The coil 50 covers at least a part of the distal end side of the second wire 40.

The coil 50 is joined to the base portion 482 of the first wire 48 via a distal-end-side bonding material 61 formed at the distal end portion of the coil 50 and a proximal-end-side bonding material 62 formed at the proximal end portion of the coil 50. The first wire 48 is connected to the coil 50 via the distal-end-side bonding material 61.

The distal-end-side bonding material 61 protrudes from a distal end 51 of the coil 50 toward the distal end side. The loop portion 481 of the first wire 48 is connected to the coil 50 via a portion of the distal-end-side bonding material 61 protruding from the distal end 51 of the coil 50. The coil 50 may be joined to the first wire 48 via a bonding material formed at an other position. The coil 50 may be joined to the second wire 40. As a material for forming the distal-end-side bonding material 61 and the proximal-end-side bonding material 62, for example, a metal solder (Au-Sn alloy, Sn-Ag alloy, Sn-Pb alloy, Pb-Ag alloy, etc.), a brazing material (aluminum alloy braze, silver braze, gold braze, etc.), an adhesive (epoxy-based adhesive, etc.), or the like is used.

The leading portion 20 has a reinforcing portion 28 located at a connection portion with the main body part 10. The connection portion of the leading portion 20 with the main body part 10 is reinforced by the reinforcing portion 28. In the present embodiment, the reinforcing portion 28 is formed of a portion of the distal-end-side bonding material 61 protruding from the distal end 51 of the coil 50 toward the distal end side. The reinforcing portion 28 may be formed by a welded portion between the leading portion 20 and the main body part 10. A constriction 29 is formed on an outer peripheral surface of the reinforcing portion 28. That is, in a part (for example, a proximal end portion) of the reinforcing portion 28, a width thereof in a direction orthogonal to the center axis Ax decreases toward a distal end of the reinforcing portion 28, and in an other part (for example, a distal end portion) of the reinforcing portion 28, a width thereof in the direction orthogonal to the center axis Ax increases toward the distal end of the reinforcing portion 28. Therefore, the outer peripheral surface of the reinforcing portion 28 has a curved surface that is recessed inward in a radial direction of the reinforcing portion 28.

As illustrated in FIG. 1, the guide wire 100 includes a first coating 31 and a second coating 32. The first coating 31 covers at least a part of the leading portion 20. The first coating 31 may cover a part of the main body part 10. In the present embodiment, the first coating 31 covers a region R31 formed by the entire leading portion 20 and the distal end portion of the main body part 10. The second coating 32 covers at least a part of the main body part 10. In the present embodiment, the second coating 32 covers a region R32 which is an intermediate portion of the main body part 10 excluding the distal end portion and the proximal end portion. The region R32 is located on a proximal end side of the region R31. A slidability of the first coating 31 is different from a slidability of the second coating 32. For example, the slidability of the first coating 31 is lower than the slidability of the second coating 32. The first coating 31 is, for example, hydrophobic and is formed of silicone. The second coating 32 is, for example, hydrophilic, and is formed of polyvinylpyrrolidone, polyacrylic acid, polyacrylamide, polyvinyl alcohol, a maleic anhydride copolymer, hyaluronic acid, or the like.

The guide wire 100 further includes a third coating 33. The third coating 33 covers at least a part of the main body part 10. In the present embodiment, the third coating 33 covers a region R33, which is the proximal end portion of the main body part 10. The region R33 is located on the proximal end side of the region R32. The third coating 33 is formed of, for example, PTFE.

In the present embodiment, as illustrated in FIG. 4, the distal end portion of the second wire 40 is disposed on the hole 482H of the base portion 482 of the first wire 48. As a result, the distal end portion of the second wire 40 and the proximal end portion of the first wire 48 are connected to each other.

In the present embodiment, as illustrated in FIG. 4, the widths of the loop portion 481 are different at respective positions around the through-hole 484 in the loop portion 481. For example, when the guide wire 100 is viewed along the X-axis direction, a shortest distance L1 from a first position P1 on an outer edge of the through-hole 484 to an outer edge of the loop portion 481 is shorter than a shortest distance L2 from a second position P2 on an outer edge of the through-hole 484 to an outer edge of the loop portion 481. The widths of the loop portion 481 may be equal at respective positions around the through-hole 484 in the loop portion 481.

In the present embodiment, as illustrated in FIG. 5, in the cross section orthogonal to the outer edge of the through-hole 484 in the loop portion 481, an inner peripheral surface 481SI of the loop portion 481 and an outer peripheral surface 481SO of the loop portion 481 are each linear. The inner peripheral surface 481SI is inclined at a predetermined angle with respect to the outer peripheral surface 481SO. In the present embodiment, the inner peripheral surface 481SI is inclined at a predetermined angle with respect to the outer peripheral surface 481SO in the XY cross section orthogonal to the axial direction of the main body part 10 among the cross sections orthogonal to the outer edge of the through-hole 484 in the loop portion 481. The predetermined angle is, for example, not less than 10° and not more than 60°. The predetermined angle may be not less than 15°and not more than 45°, or may be not less than 20°and not more than 45°. The inner peripheral surface 481SI may be parallel to the outer peripheral surface 481SO. As illustrated in FIG. 5, the loop portion 481 has an edge at an intersection IS between the outer peripheral surface 481SO of the loop portion 481 and a surface 481S intersecting the outer peripheral surface 481SO.

### Method of Manufacturing Guide Wire 100

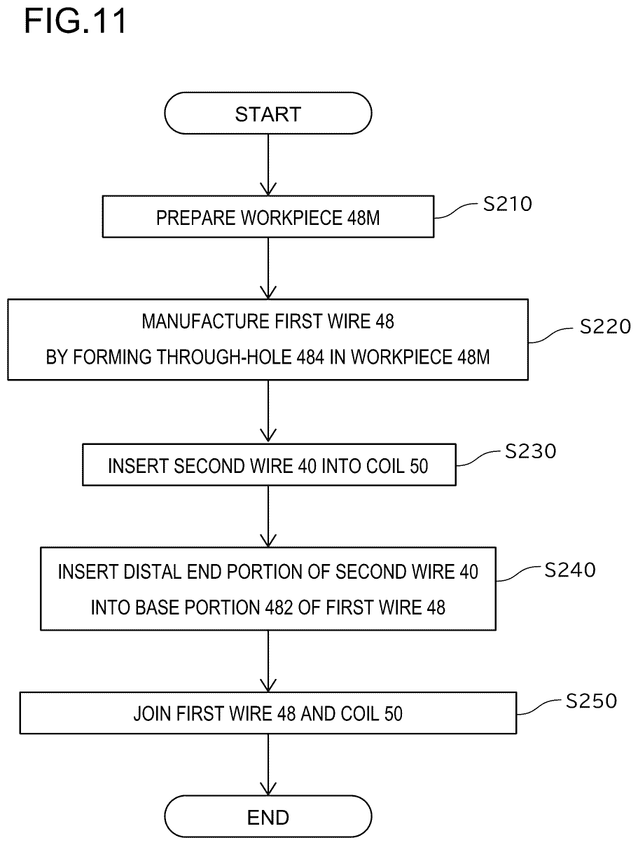

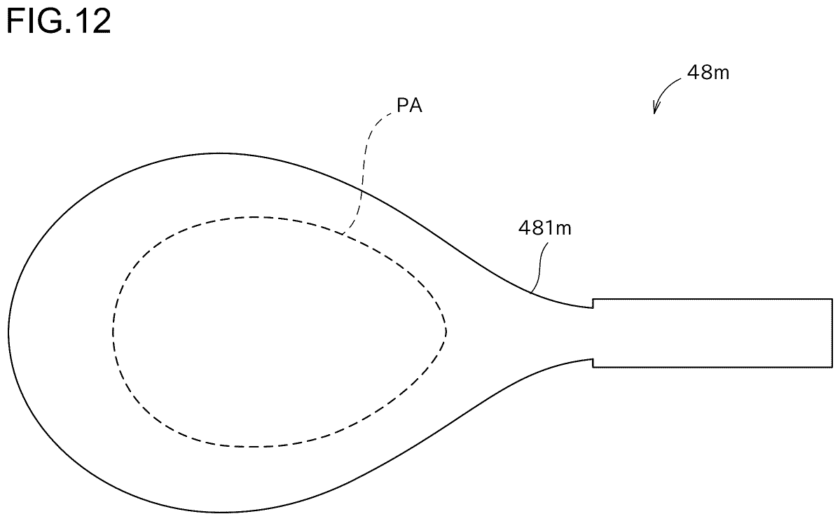

FIG. 11 is a flowchart illustrating an example of a method for manufacturing the guide wire 100. FIG. 12 is an explanatory view illustrating an example of the method for manufacturing the guide wire 100. The guide wire 100 according to the present embodiment can be manufactured, for example, by the following manufacturing method.

First, a worker prepares a workpiece 48m (S210). The workpiece 48m is a member that becomes the first wire 48 through machining. To be specific, the workpiece 48m is the first wire 48 in a state where the through-hole 484 is not formed. The worker prepares the workpiece 48m (see FIG. 12) by cutting out a plate material formed of a radiopaque material such as platinum, tantalum, rhenium, iridium, or tungsten.

Next, the worker manufactures the first wire 48 by forming the through-hole 484 in a flat portion 481m of the workpiece 48m (S220, FIG. 12). The worker may form the through-hole 484 in the flat portion 481m by cutting a machining area PA in the flat portion 481m with, for example, a drill or the like. The worker may form the through-hole 484 in the flat portion 481m by irradiating the machining area PA in the flat portion 481m with, for example, a laser. The worker manufactures the loop portion 481 by the above-described process and manufactures the first wire 48. When manufacturing the loop portion 481, the worker may form the through-hole 484 such that the widths of the loop portion 481 are different at respective positions around the through-hole 484 in the loop portion 481. To be specific, the worker may form the through-hole 484 in the flat portion 481m such that, when the guide wire 100 is viewed along the X-axis direction, the shortest distance L1 from the first position P1 on the outer edge of the through-hole 484 to the outer edge of the loop portion 481 is shorter than the shortest distance L2 from the second position P2 on the outer edge of the through-hole 484 to the outer edge of the loop portion 481. When manufacturing the loop portion 481, the worker may form the through-hole 484 such that the inner peripheral surface 481SI is inclined at a predetermined angle with respect to the outer peripheral surface 481SO.

Next, the worker inserts the second wire 40 into the coil 50 (S230). The worker prepares the second wire 40 and inserts the second wire 40 into a hollow portion of the coil 50.

Next, the worker inserts the distal end portion of the second wire 40 into the base portion 482 of the first wire 48 (S240). For example, the worker forms the hole 482H in the base portion 482 of the first wire 48 after the S220 process. The worker inserts the small diameter portion 47, which is the distal end portion of the second wire 40, into the hole 482H of the base portion 482. Thus, the first wire 48 and the second wire 40 are connected to each other. The worker may weld the first wire 48 and the second wire 40 to increase a joint strength between the first wire 48 and the second wire 40.

Next, the worker joins the first wire 48 and the coil 50 (S250). In order to join the first wire 48 and the coil 50 to each other, the worker forms the distal-end-side bonding material 61 and the proximal-end-side bonding material 62 using a bonding material such as a solder material. Thus, the first wire 48 and the coil 50 are joined to each other. At this time, the worker joins the first wire 48 and the coil 50 in such a way that the through-hole 484 is oriented in a direction intersecting the axial direction of the coil 50. The bonding material is supplied, for example, between the strands of the coil 50 to the inside of the coil 50. The reinforcing portion 28 is formed by the distal-end-side bonding material 61 protruding from the distal end of the coil 50 toward the distal end side. By the joining step, the guide wire 100 including the leading portion 20 having the reinforcing portion 28 and the main body part 10 is manufactured.

### Effect of Present Embodiment

As described above, the method for manufacturing the guide wire 100 according to the present embodiment includes: manufacturing the first wire 48 entering the lesion 220 by forming the through-hole 484 in the flat portion 481m of the workpiece 48m having the flat portion 481m, the first wire 48 having the loop portion 481 surrounding the through-hole 484; and joining the first wire 48 and the coil 50 in such a way that the through-hole 484 is oriented in the X-axis direction intersecting the axial direction of the coil 50. The method for manufacturing the guide wire 100 according to the present embodiment can manufacture the loop portion 481 having a stable structure and can improve the strength of the loop portion 481 in the guide wire 100, for example, as compared with a method for manufacturing a guide wire in which a loop portion is formed by folding back an elongated member. That is, for example, in the loop portion formed by folding back the elongated member, a position of an end portion of the elongated member is not easily determined, and thus the structure of the loop portion may be unstable. In the loop portion 481 manufactured by the method for manufacturing the guide wire 100 according to the present embodiment, the loop portion 481 can be manufactured by forming the through-hole 484 in the flat portion 481m, and therefore, the loop portion 481 having a stable structure can be manufactured. The method for manufacturing the guide wire 100 according to the present embodiment makes it possible to easily manufacture the loop portion 481 having various structures. That is, according to the method for manufacturing the guide wire 100 in the present embodiment, it is easy to manufacture the loop portion 481 into an arbitrary structure, for example, as compared with a method for manufacturing a loop portion by folding back an elongated member. To be specific, the method for manufacturing the guide wire 100 according to the present embodiment makes it easy to, for example, reduce a curvature at an arbitrary position in the loop portion 481, reduce the thickness t481, or change only the thickness at an arbitrary position in the loop portion 481.

In the method for manufacturing the guide wire 100 according to the present embodiment, the through-hole 484 is formed in the flat portion 481m by cutting the flat portion 481m. The method for manufacturing the guide wire 100 according to the present embodiment can manufacture the loop portion 481 having a stable structure and can improve the strength of the loop portion 481 in the guide wire 100, for example, as compared with a method for manufacturing a guide wire in which a loop portion is formed by folding back an elongated member.

In the method for manufacturing the guide wire 100 according to the present embodiment, the through-hole 484 is formed in the flat portion 481m by irradiating the flat portion 481m with a laser. The method for manufacturing the guide wire 100 according to the present embodiment can manufacture the loop portion 481 having a stable structure and can improve the strength of the loop portion 481 in the guide wire 100, for example, as compared with a method for manufacturing a guide wire in which a loop portion is formed by folding back an elongated member.

In the method for manufacturing the guide wire 100 according to the present embodiment, the through-hole 484 is formed in the flat portion 481m in such a way that the shortest distance L1 from the first position P1 on the outer edge of the through-hole 484 to the outer edge of the loop portion 481 is shorter than the shortest distance L2 from the second position P2 on the outer edge of the through-hole 484 to the outer edge of the loop portion 481 when the guide wire 100 is viewed along the X-axis direction. The method for manufacturing the guide wire 100 according to the present embodiment makes it possible to manufacture the loop portion 481 in which a part of the loop portion 481 is made thin and the lesion 220 can be easily drilled due to the thinned part.

In the method for manufacturing the guide wire 100 according to the present embodiment, the through-hole 484 is formed in the flat portion 481m in such a way that the inner peripheral surface 481SI and the outer peripheral surface 481SO of the loop portion 481 are each linear and the inner peripheral surface 481SI is inclined with respect to the outer peripheral surface 481SO in the cross section orthogonal to the outer edge of the through-hole 484 in the loop portion 481. In the method for manufacturing the guide wire 100 according to the present embodiment, for example, an acute edge can be formed at an intersection between the inner peripheral surface 481SI and the surface 481S by setting an angle formed by the inner peripheral surface 481SI and the surface 481S intersecting the inner peripheral surface 481SI to be smaller than 90°. Accordingly, it is possible to manufacture the loop portion 481 in which it is easy to drill the lesion 220.

The guide wire 100 according to the present embodiment includes an elongated main body part 10 and a leading portion 20 connected to the distal end 16 of the main body part 10. The leading portion 20 includes the first wire 48 that enters the lesion 220, and the first wire 48 has the loop portion 481 surrounding the through-hole 484 that passes through the first wire 48 in the X-axis direction intersecting the axial direction of the main body part 10. According to the guide wire 100 of the present embodiment, the structure of the loop portion 481 is stabilized and the strength of the loop portion 481 is improved, for example, as compared with a guide wire having a loop portion formed by folding back an elongated member. That is, for example, in the loop portion formed by folding back the elongated member, a position of an end portion of the elongated member is not easily determined, and thus the structure of the loop portion may be unstable. Since the guide wire 100 according to the present embodiment has the loop portion 481 surrounding the through-hole 484 which passes through the first wire 48, the structure of the loop portion 481 is stabilized.

In the guide wire 100 according to the present embodiment, when the guide wire 100 is viewed along the X-axis direction, the shortest distance L1 from the first position P1 on the outer edge of the through-hole 484 to the outer edge of the loop portion 481 is shorter than the shortest distance L2 from the second position P2 on the outer edge of the through-hole 484 to the outer edge of the loop portion 481. According to the guide wire 100 of the present embodiment, since a part of the loop portion 481 is thin, the lesion 220 can be easily drilled by the loop portion 481 having the thin portion.

In the guide wire 100 according to the present embodiment, in a cross section orthogonal to the outer edge of the through-hole 484 in the loop portion 481, the inner peripheral surface 481SI and the outer peripheral surface 481SO of the loop portion 481 are each linear, and the inner peripheral surface 481SI is inclined with respect to the outer peripheral surface 481SO. According to the guide wire 100 of the present embodiment, for example, the angle formed by the inner peripheral surface 481SI and the surface 481S intersecting the inner peripheral surface 481SI is smaller than 90°, and an acute edge is formed at the intersection between the inner peripheral surface 481SI and the surface 481S. This makes it easy to drill the lesion 220 by the loop portion 481.

In the guide wire 100 according to the present embodiment, the loop portion 481 has an edge at the intersection IS between the outer peripheral surface 481SO of the loop portion 481 and the surface 481S intersecting the outer peripheral surface 481SO. According to the guide wire 100 of the present embodiment, the leading portion 20 can easily drill the lesion 220.

In the guide wire 100 according to the present embodiment, the first wire 48 further includes the base portion 482 extending from the proximal end of the loop portion 481 toward the proximal end side, and the thickness t481 of the loop portion 481 in the X-axis direction is smaller than the thickness t482 of the base portion 482 in the X-axis direction. According to the guide wire 100 of the present embodiment, the leading portion 20 can easily drill the lesion 220.

In the guide wire 100 according to the present embodiment, the first wire 48 is radiopaque. According to the guide wire 100 of the present embodiment, since the first wire 48 is radiopaque, the visibility of the leading portion 20 is improved.

In the guide wire 100 of the present embodiment, the main body part 10 includes the second wire 40 whose distal end portion is connected to the proximal end portion of the first wire 48 and whose proximal end is located at the proximal end portion of the main body part 10. According to the guide wire 100 of the present embodiment, the structure of the loop portion 481 is stabilized and the strength of the loop portion 481 is improved, for example, as compared with a guide wire having a loop portion formed by folding back an elongated member.

In the guide wire 100 of the present embodiment, the first wire 48 further includes the base portion 482 extending from the proximal end of the loop portion 481 to the proximal end side, the base portion 482 in which the hole 482H extending in the axial direction of the main body part 10 is formed, and the distal end portion of the second wire 40 is disposed in the hole 482H. According to the guide wire 100 of the present embodiment, the structure of the loop portion 481 is stabilized and the strength of the loop portion 481 is improved, for example, as compared with a guide wire having a loop portion formed by folding back an elongated member.

### B. Second Embodiment

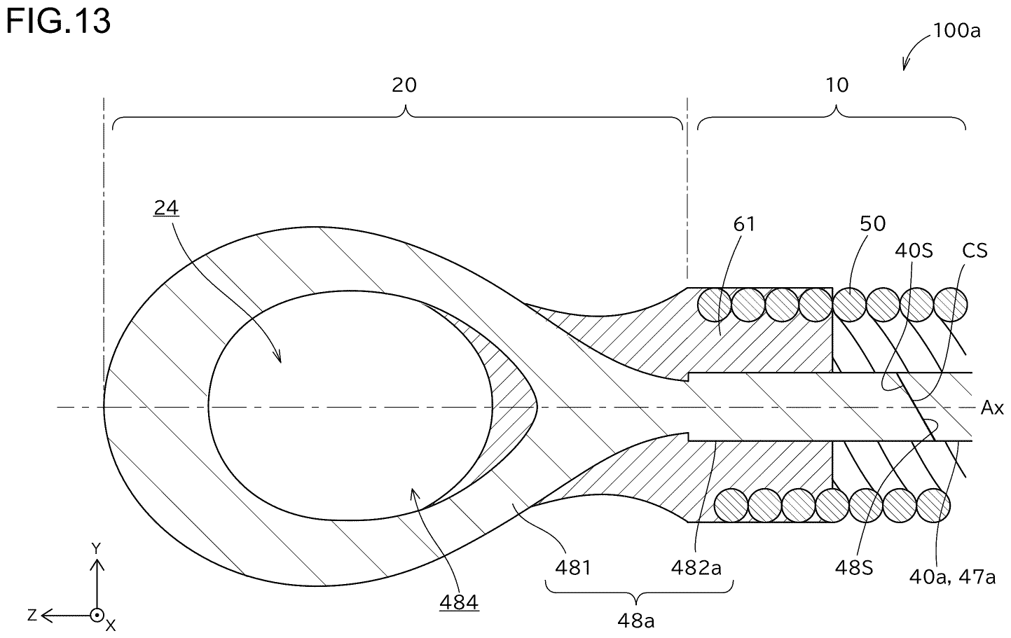

FIG. 13 is an explanatory view schematically illustrating a configuration of a guide wire 100a according to a second embodiment. FIG. 13 illustrates a YZ longitudinal section of the distal end portion of the guide wire 100a. Hereinafter, components of the guide wire 100a according to the second embodiment, which are the same as the components of the guide wire 100 according to the first embodiment, are denoted with the same reference signs, thereby omitting the description thereof as appropriate.

In the guide wire 100a according to the second embodiment, an aspect of a connection portion between a first wire 48a and a second wire 40a is different from the aspect of the connection portion between the first wire 48 and the second wire 40 in the guide wire 100 according to the first embodiment. In particular, the first wire 48a has a cross section 48S located at a proximal end portion of the first wire 48a. The cross section 48S is located at a proximal end portion of a base portion 482a in the first wire 48a. The second wire 40a has a cross section 40S located at a distal end portion of the second wire 40a. The cross section 40S is located at a distal end portion of a small diameter portion 47a of the second wire 40a. The cross section 48S is an example of a first cross section. The cross section 40S is an example of a second cross section.

The first wire 48a and the second wire 40a are connected to each other by joining the cross section 48S and the cross section 40S. The first wire 48a and the second wire 40a are joined to each other by, for example, welding. The cross section 48S and the cross section 40S are inclined at a predetermined angle with respect to a direction perpendicular to an axial direction of a main body part 10. That is, a joint surface CS between the cross section 48S and the cross section 40S is inclined at a predetermined angle with respect to the direction perpendicular to the axial direction of the main body part 10. The predetermined angle is, for example, not less than 10° and not more than 60°. The predetermined angle may be not less than 15° and not more than 45°, or may be not less than 20° and not more than 45°.

As described above, in the guide wire 100a of the present embodiment, the first wire 48a and the second wire 40a are connected to each other by joining the cross section 48S located at the proximal end portion of the first wire 48a and the cross section 40S located at the distal end portion of the second wire 40a. The joint surface CS between the cross section 48S and the cross section 40S is inclined with respect to a direction perpendicular to the axial direction of the main body part 10. According to the guide wire 100a of the present embodiment, for example, an area of the joint surface CS can be increased as compared with a guide wire in which the joint surface is not inclined with respect to a direction perpendicular to the axial direction of the main body part. The increase in the area of the joint surface CS improves a joint strength between the first wire 48a and the second wire 40a.

### C. Third Embodiment

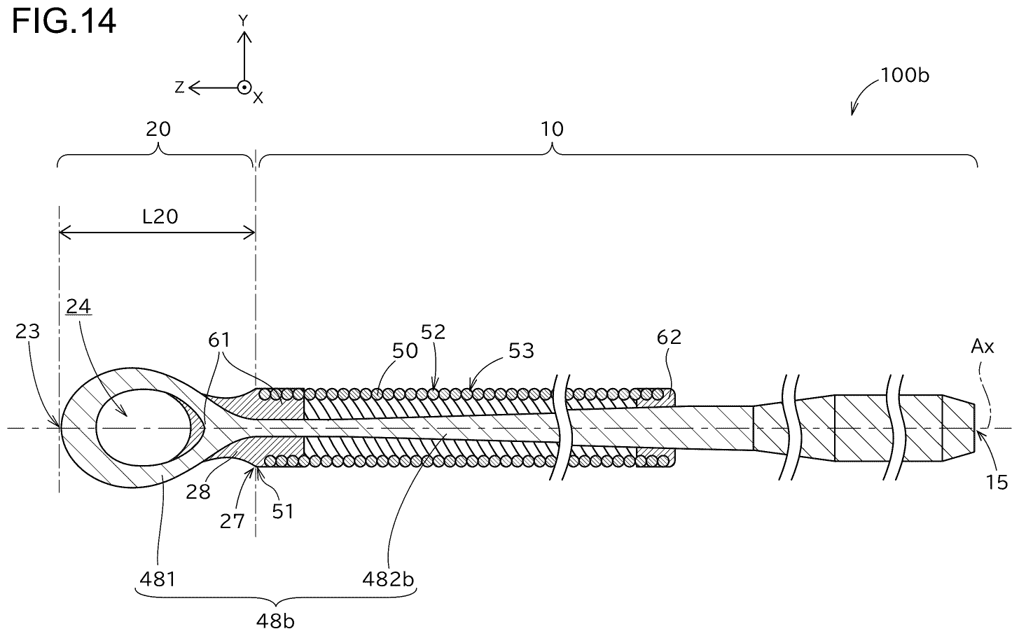

FIG. 14 is an explanatory view schematically illustrating a configuration of a guide wire 100b according to a third embodiment. FIG. 14 illustrates a YZ longitudinal section of the guide wire 100b. Hereinafter, components of the guide wire 100b according to the third embodiment, which are the same as the components of the guide wire 100 according to the first embodiment, are denoted with the same reference signs, thereby omitting the description thereof as appropriate.

In the guide wire 100b according to the third embodiment, an aspect of a first wire 48b is different from the aspect of the first wire 48 in the guide wire 100 according to the first embodiment. In detail, the first wire 48b has a base portion 482b. The base portion 482b extends from a proximal end of a loop portion 481 toward a proximal end side. The base portion 482b extends to a proximal end portion of a main body part 10. That is, the guide wire 100b does not include the second wire 40 in the first embodiment, and instead, the base portion 482b extends to the proximal end portion of the main body part 10. The coil 50 covers a distal end side of the base portion 482b. A distal end portion of the base portion 482b is connected to a distal end portion of the coil 50.

As described above, in the guide wire 100b according to the present embodiment, the first wire 48b further includes the base portion 482b extending from the proximal end of the loop portion 481 toward the proximal end side, the base portion 482b extending to the proximal end portion of the main body part 10. The main body part 10 includes a cylindrical coil 50 covering the base portion 482b. A distal end portion of the base portion 482b is connected to a distal end portion of the coil 50. According to the guide wire 100b of the present embodiment, a structure of the loop portion 481 is stabilized and a strength of the loop portion 481 is improved, for example, as compared with a guide wire having a loop portion formed by folding back an elongated member.

### D. Fourth Embodiment

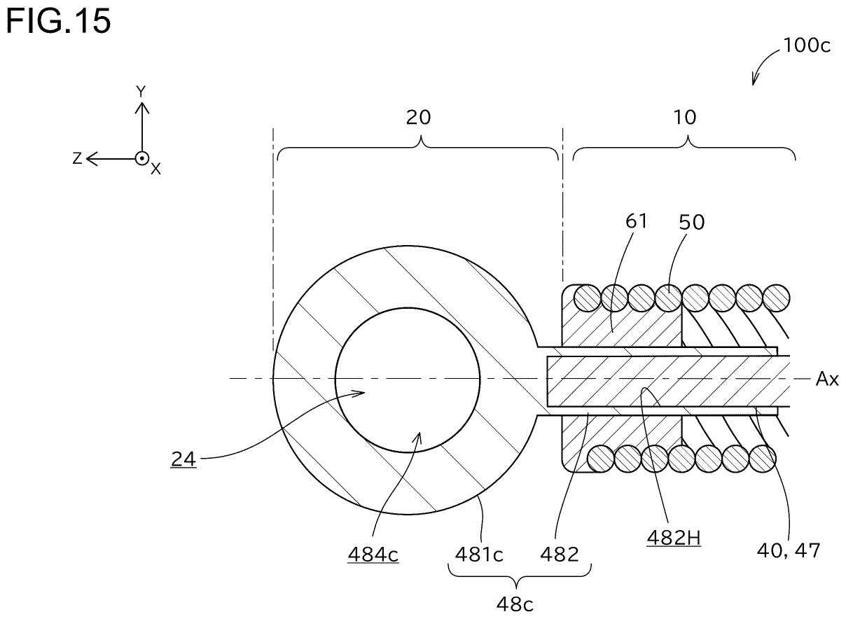

FIG. 15 is an explanatory view schematically illustrating a configuration of a guide wire 100c according to a fourth embodiment. FIG. 15 illustrates a YZ longitudinal section of a distal end portion of the guide wire 100c. Hereinafter, components of the guide wire 100c according to the fourth embodiment, which are the same as the components of the guide wire 100 according to the first embodiment, are denoted with the same reference signs, thereby omitting the description thereof as appropriate.

In the guide wire 100c according to the fourth embodiment, an aspect of a first wire 48c is different from the aspect of the first wire 48 in the guide wire 100 according to the first embodiment. The first wire 48c has a loop portion 481c. When viewed in an X-axis direction, an outer edge of the loop portion 481c is perfectly circular. When viewed in the X-axis direction, an outer edge of the through-hole 484c is perfectly circular. The first wire 48c can be manufactured in the same manner as the first wire 48 according to the first embodiment.

The method for manufacturing the guide wire 100 described in the first embodiment facilitates the manufacture of a loop portion having a perfectly circular outer edge, such as the guide wire 100c according to the present embodiment.

### E. Fifth Embodiment

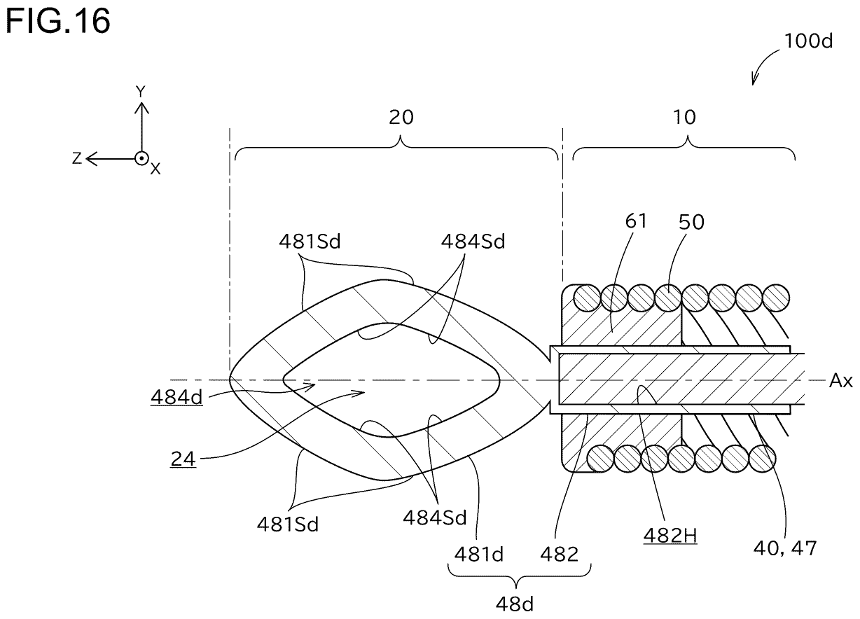

FIG. 16 is an explanatory view schematically illustrating a configuration of a guide wire 100d according to a fifth embodiment. FIG. 16 illustrates a YZ longitudinal section of a distal end portion of the guide wire 100d. Hereinafter, components of the guide wire 100d according to the fifth embodiment, which are the same as the components of the guide wire 100 according to the first embodiment, are denoted with the same reference signs, thereby omitting the description thereof as appropriate.

In the guide wire 100d according to the fifth embodiment, an aspect of a first wire 48d is different from the aspect of the first wire 48 in the guide wire 100 according to the first embodiment. The first wire 48d has a loop portion 481d. When viewed in an X-axis direction, an outer edge of the loop portion 481d is substantially rhombic. In other words, the outer edge of the loop portion 481d has four straight portions 481Sd. When viewed in the X-axis direction, an outer edge of a through-hole 484d is substantially rhombic. In other words, the outer edge of the through-hole 484d has four straight portions 484Sd. The first wire 48d can be manufactured in the same manner as the first wire 48 according to the first embodiment.

The method for manufacturing the guide wire 100 described in the first embodiment facilitates the manufacture of a loop portion having a substantially rhombic outer edge, such as the guide wire 100d according to the present embodiment.

### F. Sixth Embodiment

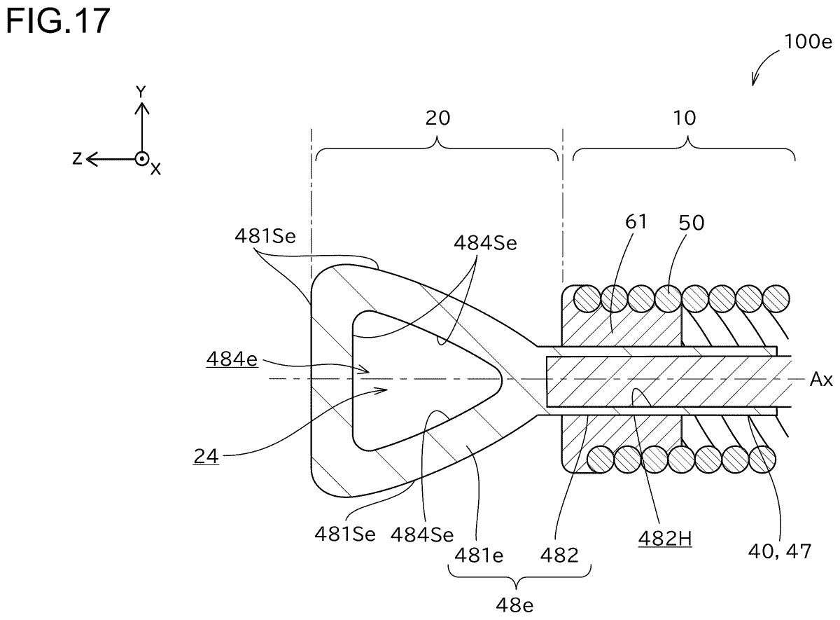

FIG. 17 is an explanatory view schematically illustrating a configuration of a guide wire 100e according to a sixth embodiment. FIG. 17 illustrates a YZ longitudinal section of a distal end portion of the guide wire 100e. Hereinafter, components of the guide wire 100e according to the sixth embodiment, which are the same as the components of the guide wire 100 according to the first embodiment, are denoted with the same reference signs, thereby omitting the description thereof as appropriate.

In the guide wire 100e according to the sixth embodiment, an aspect of a first wire 48e is different from the aspect of the first wire 48 in the guide wire 100 according to the first embodiment. The first wire 48e has a loop portion 481e. When viewed in an X-axis direction, an outer edge of the loop portion 481e is substantially triangular. In other words, the outer edge of the loop portion 481e has three straight portions 481Se. In the X-axis direction, the outer edge of a through-hole 484e is substantially triangular. In other words, the outer edge of the through-hole 484e has three straight portions 484Se. The first wire 48e can be manufactured in the same manner as the first wire 48 according to the first embodiment.

The method for manufacturing the guide wire 100 described in the first embodiment facilitates the manufacture of a loop portion having a substantially triangular outer edge, such as the guide wire 100e according to the present embodiment.

### G. Seventh Embodiment

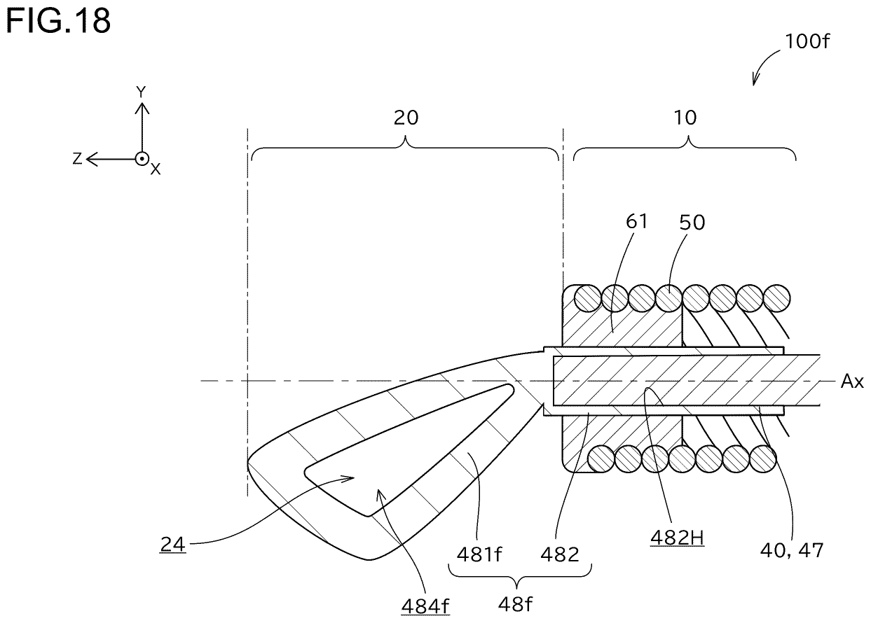

FIG. 18 is an explanatory view schematically illustrating a configuration of a guide wire 100f according to a seventh embodiment. FIG. 18 illustrates a YZ longitudinal section of a distal end portion of the guide wire 100f. Hereinafter, components of the guide wire 100f according to the seventh embodiment, which are the same as the components of the guide wire 100 according to the first embodiment, are denoted with the same reference signs, thereby omitting the description thereof as appropriate.

In the guide wire 100f according to the seventh embodiment, an aspect of a first wire 48f is different from the aspect of the first wire 48 in the guide wire 100 according to the first embodiment. The first wire 48f has a loop portion 481f. When viewed in an X-axis direction, the loop portion 481f has an asymmetric structure with respect to a center axis Ax. The loop portion 481f is located more toward a Y-axis negative direction side rather than a Y-axis positive direction side with respect to the center axis Ax. When viewed in the X-axis direction, an outer edge of a through-hole 484f has an asymmetric structure with respect to the center axis Ax. The through-hole 484f is located more toward the Y-axis negative direction side rather than the Y-axis positive direction side with respect to the center axis Ax. The first wire 48f can be manufactured in the same manner as the first wire 48 according to the first embodiment.

The method for manufacturing the guide wire 100 described in the first embodiment facilitates the manufacture of a loop portion having an asymmetric structure with respect to the center axis of a main body part, such as the guide wire 100f according to the present embodiment.

### H. Eighth Embodiment

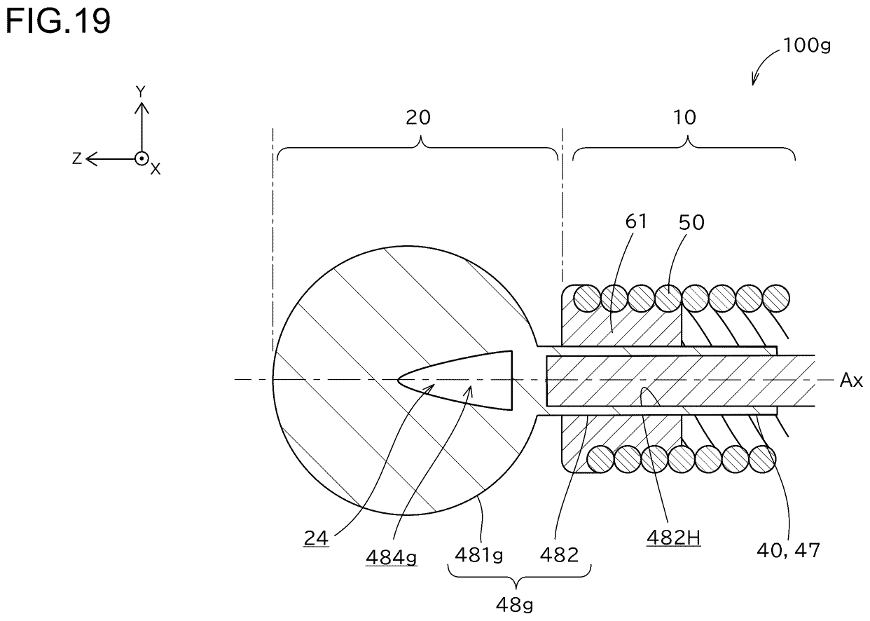

FIG. 19 is an explanatory view schematically illustrating a configuration of a guide wire 100g according to an eighth embodiment. FIG. 19 illustrates a YZ longitudinal section of a distal end portion of the guide wire 100g. Hereinafter, components of the guide wire 100g according to the eighth embodiment, which are the same as the components of the guide wire 100 according to the first embodiment, are denoted with the same reference signs, thereby omitting the description thereof as appropriate.

In the guide wire 100g according to the eighth embodiment, an aspect of a first wire 48g is different from the aspect of the first wire 48 in the guide wire 100 according to the first embodiment. The first wire 48g has a loop portion 481g. When viewed in an X-axis direction, an outer edge of the loop portion 481g is perfectly circular. When viewed in the X-axis direction, an outer edge of a through-hole 484g is partially elliptical. The partially elliptical shape is one of the two segments formed by dividing an ellipse with a chord. That is, when the guide wire 100g is viewed along the X-axis direction, the shape of the outer edge of the loop portion 481g and the shape of the outer edge of the through-hole 484g are not similar to each other. In the present embodiment, the fact that the shape of the outer edge of the loop portion 481g and the shape of the outer edge of the through-hole 484g are “not similar to each other” means that the outer edge of the loop portion 481g does not match the shape of the outer edge of the through-hole 484g even when the outer edge of the loop portion 481g is reduced. The phrase “not similar to each other” means that, for example, if the shape of the outer edge of the loop portion 481g and the shape of the outer edge of the through-hole 484g are polygonal, the ratios of corresponding sides and the angles are not equal. The first wire 48g can be manufactured in the same manner as the first wire 48 according to the first embodiment.

As described above, in the guide wire 100g of the present embodiment, when the guide wire 100g is viewed along the X-axis direction, the shape of the outer edge of the loop portion 481g and the shape of the outer edge of the through-hole 484g are not similar to each other. According to the guide wire 100g of the present embodiment, a structure of the loop portion 481g is stabilized and a strength of the loop portion 481g is improved, for example, as compared with a guide wire having a loop portion formed by folding back an elongated member.

The method for manufacturing the guide wire 100 described in the first embodiment facilitates the manufacture of a loop portion, such as the guide wire 100g according to the present embodiment, in which the shape of the outer edge of the loop portion and the shape of the outer edge of the through-hole are not similar to each other.

### I. Ninth Embodiment

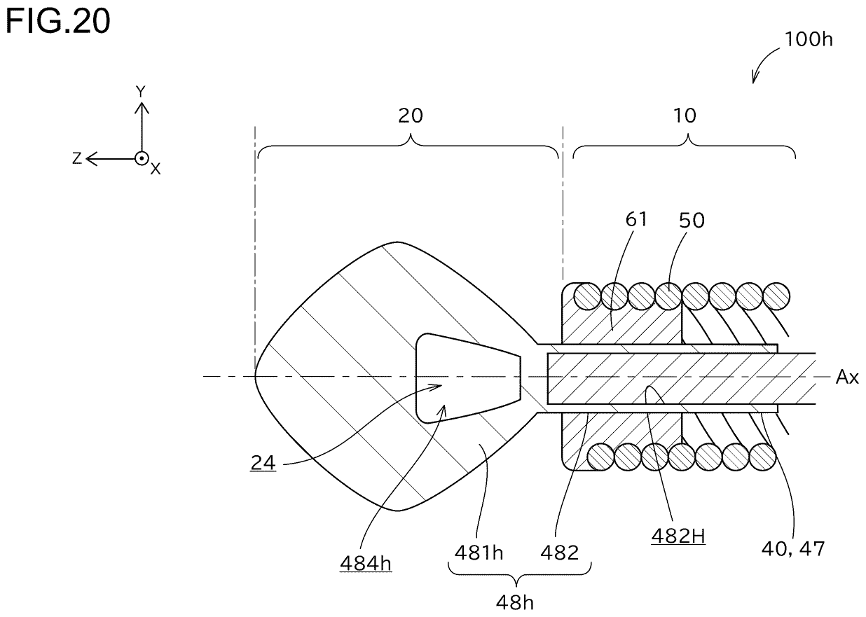

FIG. 20 is an explanatory view schematically illustrating a configuration of a guide wire 100h according to a ninth embodiment. FIG. 20 illustrates a YZ longitudinal section of a distal end portion of the guide wire 100h. Hereinafter, components of the guide wire 100h according to the ninth embodiment, which are the same as the components of the guide wire 100 according to the first embodiment, are denoted with the same reference signs, thereby omitting the description thereof as appropriate.

In the guide wire 100h according to the ninth embodiment, an aspect of the first wire 48h is different from the aspect of the first wire 48 in the guide wire 100 of the first embodiment. The first wire 48h has a loop portion 481h. When viewed in an X-axis direction, an outer edge of the loop portion 481h is substantially rhombic. When viewed in the X-axis direction, an outer edge of a through-hole 484h is substantially trapezoidal. That is, when the guide wire 100h is viewed along the X-axis direction, the shape of the outer edge of the loop portion 481h and the shape of the outer edge of the through-hole 484h are not similar to each other. The first wire 48h can be manufactured in the same manner as the first wire 48 according to the first embodiment.

The method for manufacturing the guide wire 100 described in the first embodiment facilitates the manufacture of a loop portion, such as the guide wire 100h according to the present embodiment, in which the shape of the outer edge of the loop portion and the shape of the outer edge of the through-hole are not similar to each other.

### J. Tenth Embodiment

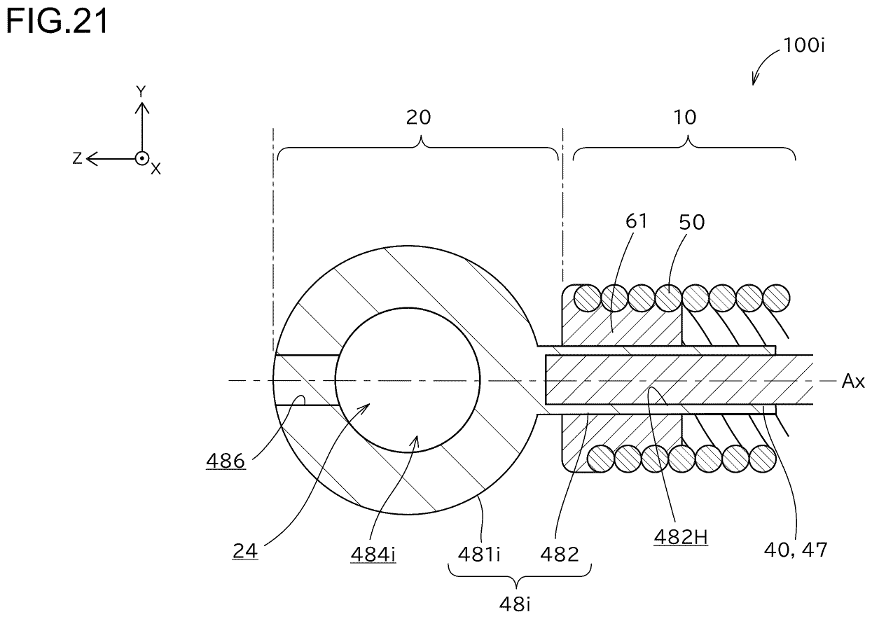

FIG. 21 is an explanatory view schematically illustrating a configuration of a guide wire 100i according to a tenth embodiment. FIG. 21 illustrates a YZ longitudinal section of a distal end portion of the guide wire 100i. Hereinafter, components of the guide wire 100i according to the tenth embodiment, which are the same as the components of the guide wire 100 according to the first embodiment, are denoted with the same reference signs, thereby omitting the description thereof as appropriate.

In the guide wire 100i according to the tenth embodiment, an aspect of a first wire 48i is different from the aspect of the first wire 48 in the guide wire 100 according to the first embodiment. The first wire 48i has a loop portion 481i. When viewed in an X-axis direction, an outer edge of the loop portion 481i is perfectly circular. When viewed in the X-axis direction, an outer edge of a through-hole 484i is perfectly circular. A groove 486 is formed in the loop portion 481i. The groove 486 is formed on a surface of the loop portion 481i facing the X-axis direction intersecting the axial direction of the main body part 10. The groove 486 extends from the outer edge of the through-hole 484i to the outer edge of the loop portion 481i. The first wire 48i can be manufactured in the same manner as the first wire 48 according to the first embodiment. A worker can form the groove 486 in the loop portion 481i by cutting the surface of the loop portion 481i. The worker can form the groove 486 in the loop portion 481i by irradiating the surface of the loop portion 481i with a laser.

The method for manufacturing the guide wire 100 described in the first embodiment facilitates the manufacture of a loop portion with a groove formed therein, such as the guide wire 100i according to the present embodiment.

### K. Modifications

The technology disclosed in this specification is not limited to the embodiments described above, and can be modified in various forms without departing from the gist of this specification, and, for example, the following modifications can be made.

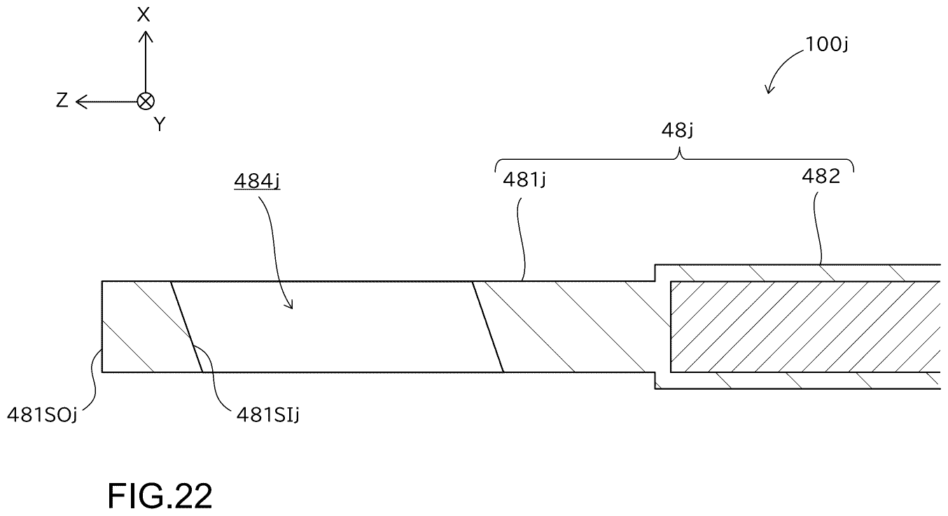

FIG. 22 is an explanatory view schematically illustrating a configuration of a guide wire 100j according to a modification. FIG. 22 illustrates an XZ longitudinal section of the guide wire 100j at the same location as the XZ longitudinal section of the guide wire 100 illustrated in FIG. 6. Hereinafter, components of the guide wire 100j according to the modification, which are the same as the components of the guide wire 100 according to the first embodiment, are denoted with the same reference signs, thereby omitting the description thereof as appropriate.

In the guide wire 100j according to the modification, a through-hole 484j is formed in a loop portion 481j of a first wire 48j. As illustrated in FIG. 22, in the XZ cross section, an inner peripheral surface 481SIj of the loop portion 481j and an outer peripheral surface 481SOj of the loop portion 481j are each linear. The inner peripheral surface 481SIj is inclined at a predetermined angle with respect to the outer peripheral surface 481SOj. In this way, in a cross section parallel to the axial direction of the main body part among cross sections orthogonal to an outer edge of the through-hole in the loop portion, the inner peripheral surface of the loop portion may be inclined with respect to the outer peripheral surface of the loop portion. The inner peripheral surface of the loop portion may be inclined with respect to the outer peripheral surface of the loop portion over the entire circumference of the loop portion.

The configuration of the guide wire 100 according to the above-described embodiment is merely an example and may be modified in various ways. For example, the guide wire may not include at least one of the first coating 31, the second coating 32, and the third coating 33.

The method for manufacturing the guide wire 100 according to each of the above-described embodiments is merely an example and can be modified in various ways.

For example, the method for forming the through-hole in the flat portion is not limited to cutting and laser irradiation.

The material for each member according to the above-described embodiments is merely an example and may be variously modified. The treatment method using the guide wire 100 in the above-described embodiment is merely an example, and can be modified in various ways.

In the above-described embodiment, the guide wire 100 for treating a lesion in a blood vessel has been described as an example. The technology disclosed in this specification is similarly applicable to medical devices in general for treating the lesion in a living body lumen.

Each of all the features described in each of the embodiments described above may be appropriately combined with an other embodiment, or may be appropriately combined with the modification. Each of all the features described in each of the modifications described above may be appropriately combined with the embodiment or may be appropriately combined with an other modification. Each of all the features described in each of the embodiments described above may be omitted as appropriate. Each of all the features described in each of the modifications described above may be omitted as appropriate.

1. A method for manufacturing a medical device, comprising:
manufacturing an entry member entering a lesion by forming a through-hole in a flat portion of a workpiece having the flat portion, the entry member having a loop portion surrounding the through-hole; and
joining the entry member and an elongated body to each other in such a way that the through-hole is oriented in a predetermined direction intersecting an axial direction of the elongated body.

2. The method for manufacturing the medical device according to claim 1, wherein
the through-hole is formed in the flat portion by cutting the flat portion.

3. The method for manufacturing the medical device according to claim 1, wherein
the through-hole is formed in the flat portion by irradiating the flat portion with a laser.

4. The method for manufacturing the medical device according to claim 1, wherein,
when the medical device is viewed along the predetermined direction, the through-hole is formed in the flat portion in such a way that a shortest distance from a first position on an outer edge of the through-hole to an outer edge of the loop portion is shorter than a shortest distance from a second position on an outer edge of the through-hole to an outer edge of the loop portion.

5. The method for manufacturing the medical device according to claim 1, wherein
the through-hole is formed in the flat portion in such a way that in a cross section orthogonal to an outer edge of the through-hole in the loop portion,
an inner peripheral surface and an outer peripheral surface of the loop portion are each linear, and
the inner peripheral surface is inclined with respect to the outer peripheral surface.

6. A medical device comprising:
an elongated main body part; and
a leading portion connected to a distal end of the main body part,
the leading portion including an entry member entering a lesion,
the entry member having a loop portion surrounding a through-hole passing through the entry member in a predetermined direction intersecting an axial direction of the main body part.

7. The medical device according to claim 6, wherein,
when the medical device is viewed along the predetermined direction, a shortest distance from a first position on an outer edge of the through-hole to an outer edge of the loop portion is shorter than a shortest distance from a second position on an outer edge of the through-hole to an outer edge of the loop portion.

8. The medical device according to claim 6, wherein
in a cross section orthogonal to an outer edge of the through-hole in the loop portion,
an inner peripheral surface and an outer peripheral surface of the loop portion are each linear, and
the inner peripheral surface is inclined with respect to the outer peripheral surface.

9. The medical device according to claim 6, wherein
in a cross section orthogonal to an outer edge of the through-hole in the loop portion, the loop portion has an edge at an intersection between an outer peripheral surface of the loop portion and a surface intersecting the outer peripheral surface.

10. The medical device according to claim 6, wherein,
when the medical device is viewed along the predetermined direction, a shape of an outer edge of the loop portion and a shape of an outer edge of the through-hole are not similar to each other.

11. The medical device according to claim 6, wherein
the entry member further includes a base portion extending from a proximal end of the loop portion toward a proximal end side, and
a thickness of the loop portion in the predetermined direction is smaller than a thickness of the base portion in the predetermined direction.

12. The medical device according to claim 6, wherein
the entry member is radiopaque.

13. The medical device according to claim 6, wherein
the entry member is a first wire and
the main body part includes a second wire whose distal end portion is connected to a proximal end portion of the first wire and whose proximal end is located at a proximal end portion of the main body part.

14. The medical device according to claim 13, wherein
the first wire further includes a base portion extending from a proximal end of the loop portion toward a proximal end side, the base portion having a hole formed therein, the hole extending in an axial direction of the main body part, and
a distal end portion of the second wire is disposed in the hole.

15. The medical device according to claim 13, wherein
the first wire and the second wire are connected to each other by joining a first cross section located at a proximal end portion of the first wire and a second cross section located at a distal end portion of the second wire to each other, and
a joint surface between the first cross section and the second cross section is inclined with respect to a direction perpendicular to an axial direction of the main body part.

16. The medical device according to claim 6, wherein
the entry member further includes a base portion extending from a proximal end of the loop portion toward a proximal end side, the base portion extending to a proximal end portion of the main body part,
the main body part includes a cylindrical elongated body covering the base portion, and
a distal end portion of the base portion is connected to a distal end portion of the elongated body.
